(19) United States
(12) Patent Application Publication
(10) Pub. No.: US 2026/0000334 A1
(43) Pub. Date: Jan. 1, 2026

(54) METHOD FOR PROVIDING NECESSARY INFORMATION FOR ARRHYTHMIA CLASSIFICATION AND DIAGNOSIS USING 12-LEAD ELECTROCARDIOGRAM SIGNAL AND APPARATUS FOR EXECUTING THE METHOD

(71) Applicant: AJOU UNIVERSITY INDUSTRY-ACADEMIC COOPERATION FOUNDATION, Gyeonggi-do (KR)

(72) Inventors: MYUNG HOON SUNWOO, Seoul (KR); HO KEUN KIM, Gyeonggi-do (KR); JUNG WON LEE, Seoul (KR)

(21) Appl. No.: 19/077,346

(22) Filed: Mar. 12, 2025

(30) Foreign Application Priority Data
Jul. 1, 2024 (KR) 10-2024-0086457

(51) Int. Cl. A61B 5/346; A61B 5/00; G16H 50/20

(52) U.S. Cl. CPC A61B 5/346; A61B 5/7264; G16H 50/20

(57) ABSTRACT

A method for providing necessary information for arrhythmia classification and diagnosis using a 12-lead electrocardiogram signal includes obtaining a 12-lead electrocardiogram signal, outputting an analysis result for the 12-lead electrocardiogram signal using a machine learning-based technology from the 12-lead electrocardiogram signal, and generating information necessary for arrhythmia classification and diagnosis based on the analysis result.

### CROSS-REFERENCE TO RELATED APPLICATION AND CLAIM OF PRIORITY

This application claims the benefit under 35 USC § 119 of Korean Patent Application No. 10-2024-0086457 filed on Jul. 1, 2024, in the Korean Intellectual Property Office, the entire disclosure of which is incorporated herein by reference for all purposes.

### BACKGROUND

### 1. Technical Field

Embodiments of the present disclosure relate to a technology for providing information necessary for arrhythmia classification and diagnosis using a 12-lead electrocardiogram signal.

### 2. Description of Related Art

According to the World Health Organization (WHO), millions of people around the world die annually due to heart disease. Electrocardiography, known as a method for examining the presence or absence of heart disease, is a noninvasive and most commonly known examination method that detects and records a state of electrical activity in the heart during a heartbeat cycle by attaching electrodes to the skin. Generally, 12 electrocardiogram signals are obtained and analyzed using 10 electrodes attached to the arms, legs, and chest to determine the presence or absence of heart disease such as arrhythmia.

Previously, doctors diagnosed heart disease through recording of the electrocardiogram signals recorded for each patient, but recently, attempts to use deep learning for analysis and diagnosis of electrocardiogram signals are increasing.

In the existing deep learning technology for analyzing and diagnosing electrocardiogram signals, cardiac arrhythmias were classified by extracting time-frequency features from single-lead electrocardiogram signals, but, since some cardiac arrhythmias are observed only in specific electrocardiogram channels, the classification accuracy may be reduced depending on the type of cardiac arrhythmia to be classified.

Therefore, in order to accurately diagnose cardiac arrhythmias, it is necessary to comprehensively and closely check the electrocardiogram signals obtained from 12 channels. That is, since the electrocardiogram signals measured through 12 leads differ depending on the type of arrhythmia, it is necessary to comprehensively analyze the electrocardiogram signals obtained from 12 channels in order to accurately determine the patient's arrhythmia.

Examples of related art include Republic of Korea registered patent publication No. 10-2163217 (Sep. 29, 2020).

### SUMMARY

Embodiments of the present disclosure are intended to provide information necessary for arrhythmia classification and diagnosis from a 12-lead electrocardiogram signal using machine learning technology.

According to an exemplary embodiment of the present disclosure, there is provided a method for providing necessary information for arrhythmia classification and diagnosis using a 12-lead electrocardiogram signal performed by a computing device including one or more processors and a memory storing one or more programs executed by the one or more processors, the method including obtaining a 12-lead electrocardiogram signal, outputting an analysis result for the 12-lead electrocardiogram signal using a machine learning-based technology from the 12-lead electrocardiogram signal, and generating information necessary for arrhythmia classification and diagnosis based on the analysis result.

In the outputting of the classification result, the 12-lead electrocardiogram signal may be received through a pre-trained artificial neural network model and a type of arrhythmia may be classified based on the 12-lead electrocardiogram signal.

The classifying of the arrhythmia information may further include outputting, through an initial feature block, an initial feature map through a convolution operation based on the input 12-lead electrocardiogram signal, outputting, through an attention block, a focused feature map through an element-wise weighting operation based on the output initial feature map, outputting, through a residual block, a deep feature map through a shortcut operation based on the output initial feature map, summing, through a sum block, the output focused feature map and the output deep feature map to output a final feature map, and estimating a probability of a preset class based on the output final feature map and classifying a type of arrhythmia based on the output final feature map, through a classification block, and the information required for the arrhythmia classification and diagnosis may include the type of the classified arrhythmia, the estimated probability, and the input 12-lead electrocardiogram signal.

The outputting of the focused feature map may further include performing maximum pooling and average pooling in parallel on the initial feature map through a maximum pooling layer and an average pooling layer, performing summation of pooling results, which are respectively output through the maximum pooling layer and the average pooling layer, through a first summation layer to output a weighted feature map, and performing summation between the initial feature map and the weighted feature map through a second summation layer to output a focused feature map.

The residual block may be configured to include N (N is a natural number greater than or equal to 2) short residual blocks sequentially connected to reflect features of the initial feature map, and the N short residual blocks may be configured to receive a previous feature map (a feature map output from an (N-1)-th short residual block), output a new feature map from the previous feature map through a convolution layer, and sums the previous feature map and the new feature map through a third summation layer to output the summation result.

The first summation layer and the third summation layer may use element-wise sum, and the second summation layer may use element-wise multiplication.

The classifying of the type of arrhythmia may further include performing global max pooling and global average pooling in parallel on the final feature map through a global max pooling layer and a global average pooling layer and performing concatenation of pooling results, which are respectively output through the global max pooling layer and the global average pooling layer, through the connection layer.

According to another exemplary embodiment of the present disclosure, there is provided a computing device including one or more processors and a memory storing one or more programs executed by the one or more processors, the computing device including a signal obtaining module configured to obtain a 12-lead electrocardiogram signal, a signal analysis module configured to output an analysis result for the 12-lead electrocardiogram signal using a machine learning-based technology from the 12-lead electrocardiogram signal, and a result provision module configured to generate information necessary for arrhythmia classification and diagnosis based on the analysis result.

### BRIEF DESCRIPTION OF THE DRAWINGS

FIG. 1 is a block diagram for describing a configuration of an apparatus for providing information necessary for arrhythmia classification and diagnosis using a 12-lead electrocardiogram signal according to an embodiment of the present disclosure.

FIG. 2 is a block diagram for describing a configuration of a signal analysis module constituting the apparatus for providing information necessary for arrhythmia classification and diagnosis using the 12-lead electrocardiogram signal according to an embodiment of the present disclosure.

FIG. 3 is a diagram schematically illustrating a structure of the signal analysis module constituting the apparatus for providing information necessary for arrhythmia classification and diagnosis using the 12-lead electrocardiogram signal according to an embodiment of the present disclosure.

FIG. 4 is a diagram illustrating a screen of a smart device on which an analysis result provided by a result provision module of the apparatus for providing information necessary for arrhythmia classification and diagnosis using the 12-lead electrocardiogram signal according to an embodiment of the present disclosure is displayed.

FIG. 5 is a diagram for describing an attention block of the apparatus for providing information necessary for arrhythmia classification and diagnosis using the 12-lead electrocardiogram signal according to an embodiment of the present disclosure.

FIG. 6 is a diagram for describing a residual block of the apparatus for providing information necessary for arrhythmia classification and diagnosis using the 12-lead electrocardiogram signal according to an embodiment of the present disclosure.

FIG. 7 is a flowchart for describing a method for providing information necessary for arrhythmia classification and diagnosis using a 12-lead electrocardiogram signal according to an embodiment of the present disclosure.

FIG. 8 is a block diagram for illustratively describing a computing environment including a computing device suitable for use in exemplary embodiments.

### DETAILED DESCRIPTION

Hereinafter, a specific embodiment of the present disclosure will be described with reference to the drawings. The following detailed description is provided to aid in a comprehensive understanding of the methods, apparatus and/or systems described herein. However, this is illustrative only, and the present disclosure is not limited thereto.

In describing the embodiments of the present disclosure, when it is determined that a detailed description of related known technologies may unnecessarily obscure the subject matter of the present disclosure, a detailed description thereof will be omitted. Additionally, terms to be described later are terms defined in consideration of functions in the present disclosure, which may vary according to the intention or custom of users or workers. Therefore, the definition should be made based on the contents throughout this specification. The terms used in the detailed description are only for describing embodiments of the present disclosure, and should not be limiting. Unless explicitly used otherwise, expressions in the singular form include the meaning of the plural form. In this description, expressions such as “comprising” or “including” are intended to refer to certain features, numbers, steps, actions, elements, some or combination thereof, and it is not to be construed to exclude the presence or possibility of one or more other features, numbers, steps, actions, elements, some or combinations thereof, other than those described.

In the description below, the terms “transfer”, “communication”, “transmission”, “reception”, and other similar meanings of signals or information include not only direct transmission of signals or information from one component to another, but also transmission via another component. In particular, “transferring” or “transmitting” a signal or information to a component indicates the final destination of the signal or information and does not mean the direct destination. The same applies to “receiving” a signal or information. In addition, in this specification, the fact that two or more pieces of data or information are “related” means that when one piece of data (or information) is obtained, at least a part of the other data (or information) can be obtained based on it.

Meanwhile, the embodiment of the present disclosure may include a program for performing the methods described in this specification on a computer, and a computer-readable recording medium including the program. The computer-readable recording medium may include program instructions, local data files, local data structures, etc., alone or in combination. The medium may be one that is specifically designed and configured for the present disclosure, or one that is commonly available in the field of computer software. Examples of the computer-readable recording medium include hardware devices such as magnetic media, such as hard disks, floppy disks, and magnetic tapes, optical recording media, such as CD-ROMs and DVDs, ROMs, RAMs, and flash memories that are specifically configured to store and perform program commands. Examples of the program may include not only machine language codes such as those produced by a compiler, but also high-level language codes that can be executed by a computer using an interpreter, etc.

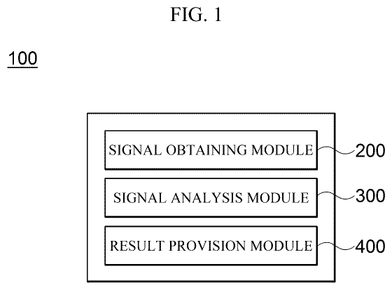

FIG. 1 is a block diagram for describing a configuration of an apparatus for providing information necessary for arrhythmia classification and diagnosis using a 12-lead electrocardiogram signal according to one embodiment of the present disclosure.

First, the configurations according to the embodiment of the present invention may be operated in a form in which applications programs each performing a function are installed and executed on a single computer or server, rather than having a physical entity, or may be operated in a form in which application programs performing one or more functions of configurations are installed on a plurality of servers, rather than having a physical entity, and organically operated through an open network.

The server has the same configuration as a typical web server in terms of hardware. However, in terms of software, the server includes program modules that are implemented in any language such as C, C++, Java, Visual Basic, and Visual C and perform various functions.

In addition, the computer or server on which the configurations described above are installed may be implemented in the form of a web server, and the web server generally means a computer system that is connected to an unspecified number of clients and/or other servers through an open computer network such as the Internet, receives a task execution request from a client or other web server, derives a task result for the request, and provides the task result, and computer software (web server program) installed for the computer system.

However, the web server should be understood as a broad concept that includes, in addition to the aforementioned web server program, a series of application programs running on the web server, and, in some cases, various databases built in the inside thereof.

In one embodiment, in a smart device of a user (e.g., a medical staff), an application for providing a service provided by an apparatus for providing information necessary for arrhythmia classification and diagnosis using the 12-lead electrocardiogram signal may be installed. The application may be stored in a computer-readable storage medium of the smart device. The application includes a predetermined set of instructions executable by a processor of the smart device. The instructions may cause the processor of the smart device to perform operations according to an exemplary embodiment. A computer-readable storage medium of the smart device includes components of an operating system for executing a set of instructions such as the application on the smart device. For example, such an operating system may be iOS from Apple or Android from Google.

As illustrated in FIG. 1, an apparatus 100 for providing information necessary for arrhythmia classification and diagnosis using a 12-lead electrocardiogram signal according to one embodiment of the present disclosure may include a signal obtaining module 200, a signal analysis module 300, and a result provision module 400.

In one embodiment, the signal obtaining module 200, the signal analysis module 300, and the result provision module 400 may be implemented using one or more physically separated devices, or may be implemented by one or more processors or a combination of one or more processors and software, and may not be clearly separated in specific operations, unlike the illustrated example.

In addition, in this specification, a module may refer to a functional and structural combination of hardware for carrying out the technical idea of the present invention and software for driving the hardware. For example, the “module” described above may mean a logical unit of a given code and hardware resources for executing the given code, and does not necessarily mean physically connected code or one type of hardware.

The signal obtaining module 200 may obtain a 12-lead electrocardiogram signal. For example, the signal obtaining module 200 may obtain a 12-lead electrocardiogram signal measured by a medical staff through a 12-lead electrocardiogram device.

Meanwhile, the 12-lead electrocardiogram signal may be obtained using 10 skin surface sensors including 4 limb leads (right arm (RA), left arm (LA), right leg (RL), and left leg (LL)) and 6 chest leads (V1, V2, V3, V4, V5, and V6). The 12-lead electrocardiogram signal has a characteristic in which a signal form in each lead varies depending on the type of arrhythmia, and abnormal signs are observed only in a specific lead among the 12 leads.

The signal obtaining module 200 may provide the obtained 12-lead electrocardiogram signal to the signal analysis module 300.

The signal analysis module 300 may output analysis results for the 12-lead electrocardiogram signal from the 12-lead electrocardiogram signal using a machine learning-based technology. For example, the signal analysis module 300 may include an artificial neural network model that has been trained to classify arrhythmia based on the input 12-lead electrocardiogram signal. In this case, the artificial neural network model may be a lightweight model after being trained on a deep learning network learning server. That is, the artificial neural network model may be a lightweight artificial neural network model that has been trained to be operated on an application installed on a smart device.

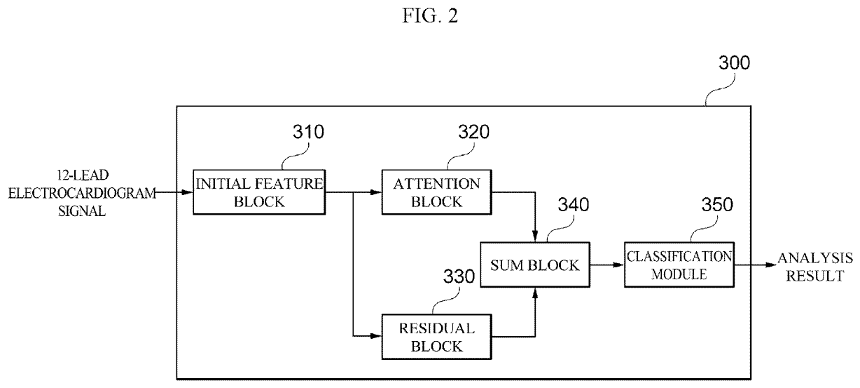

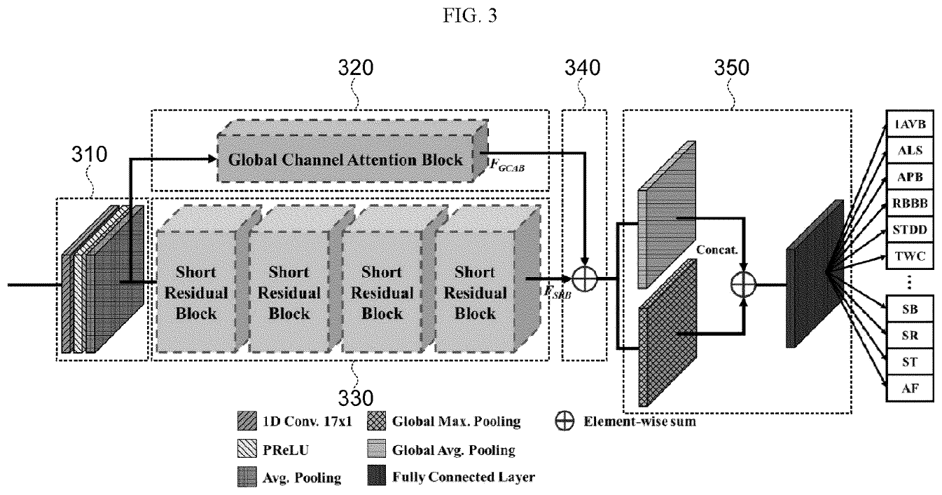

Meanwhile, a detailed description of the operation and configuration of the signal analysis module 300 will be described below with reference to FIGS. 2 and 3.

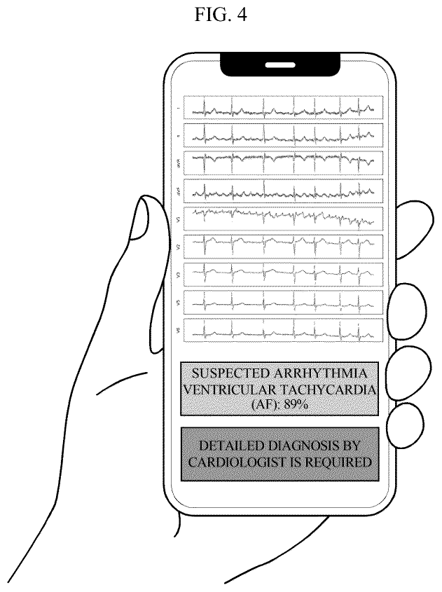

The result provision module 400 may generate information necessary for arrhythmia classification and diagnosis based on the analysis results output from the signal analysis module 300 and provide the information to the user (e.g., the medical staff). Specifically, the result provision module 400 may generate information necessary for arrhythmia classification and diagnosis including types of arrhythmia classified based on analysis results, estimated probability for the type of arrhythmia, and the input 12-lead electrocardiogram signals. In this case, the 12-lead electrocardiogram signal may be in a state where the PQRST wave is emphasized. For example, the result provision module 400 may provide the user with a 12-lead electrocardiogram signal by displaying it on a display unit of the smart device, along with the type of arrhythmia that the patient will be diagnosed with and the probability of being diagnosed with the arrhythmia, based on the analysis results, as shown in FIG. 4. That is, by providing the 12-lead electrocardiogram signal (with the PQRST wave emphasized), which is the basis for arrhythmia classification and diagnosis, along with the type of arrhythmia and the probability of being diagnosed with the arrhythmia, the problem of reduced accuracy due to the lightweight artificial neural network model can be solved.

Therefore, the apparatus for providing information necessary for arrhythmia classification and diagnosis using the 12-lead electrocardiogram signal according to one embodiment of the present invention can reduce the time required for a medical staff (cardiologist) to determine arrhythmia by providing information necessary for arrhythmia classification and diagnosis from a standard 12-lead electrocardiogram signal to a smart device using machine learning technology.

FIG. 2 is a block diagram for describing a configuration of a signal analysis module constituting the apparatus for providing information necessary for arrhythmia classification and diagnosis using the 12-lead electrocardiogram signal according to one embodiment of the present invention and FIG. 3 is a diagram schematically illustrating a structure of the signal analysis module constituting a device providing information necessary for arrhythmia classification and diagnosis using the 12-lead electrocardiogram signal according to one embodiment of the present invention.

The signal analysis module 300 illustrated in FIGS. 2 and 3 may include an initial feature block 310, an attention block 320, a residual block 330, a sum block 340, and a classification block 350.

The initial feature block 310 may output an initial feature map through a convolution operation based on an input 12-lead electrocardiogram signal.

In an exemplary embodiment, the initial feature block 310 may include a convolution layer, an activation layer (PReLU), and an average pooling layer. That is, the initial feature block 310 may output an initial feature map from the 12-lead electrocardiogram signal through the convolution layer, the activation layer, and the average pooling layer. Here, the activation layer may use the parametric ReLU (PReLU) function as an activation function. The activation function is necessary to readjust the signal strength of neurons, and the PReLU function is a function that outputs a value less than 0 by multiplying the value by a parameter (a) adjusted through training and outputs a value greater than 0 as it is.

The attention block 320 may output a focused feature map through an element-wise weighting operation based on the initial feature map output from the initial feature block 310.

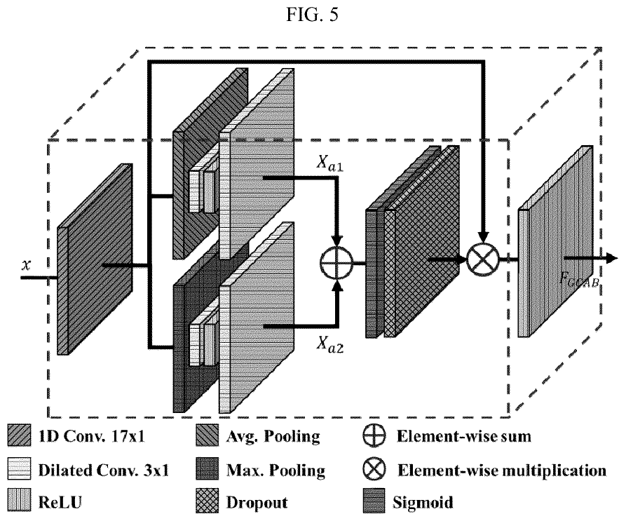

In an exemplary embodiment, as illustrated in FIG. 5, the attention block 320 may include a convolution layer, a max pooling layer, an average pooling layer, a plurality of dilated convolution layers, a plurality of activation layers, and a plurality of summation layers.

Specifically, the attention block 320 may perform max pooling and average pooling in parallel and simultaneously through a max pooling layer and an average pooling layer. In addition, the attention block 320 may perform an extended convolution operation on pooling results which are respectively produced through the max pooling layer and the average pooling layer, and perform an operation (element-wise sum) that sums the performance results through a first summation layer. Here, a value output as a result of the operation may be a weighted feature map (i.e., element-wise weight value). In addition, the attention block 320 may perform an operation (element-wise multiplication) that sums the initial feature map and the weighted feature map through a second summation layer. In addition, the attention block 320 may output a focused feature map through an activation function (ReLU) based on the operation result. In this case, the attention block 320 may prevent overfitting by making the weighted feature map have a range of element-wise weight values from 0 to 1 through an activation layer (sigmoid function) and dropout before performing the operation through the second summation layer.

That is, the attention block 320 considers a distance between elements using max pooling, average pooling, and extended convolution operations, assigns a weight value to each element according to which element is important, and outputs a weighted feature map, and may emphasize important elements among the elements through the summation of the weighted feature map and the initial feature map. Here, the elements may be pixels of the feature map. Meanwhile, in the present disclosure, an interval (dilation rate) of the extended convolution layer is set to 2, but is not limited thereto.

The residual block 330 may output a deep feature map through a shortcut operation based on the initial feature map output from the initial feature block 310.

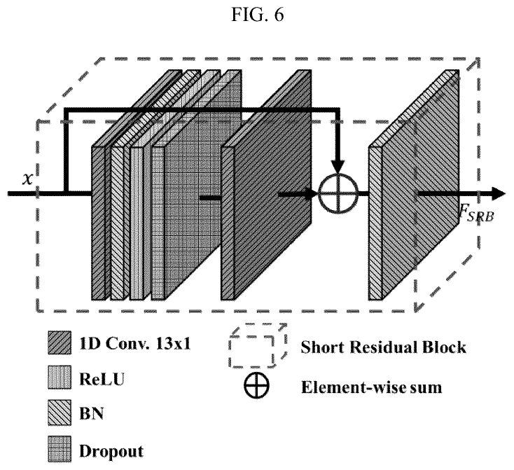

In an exemplary embodiment, the residual block 330 may include a plurality of short residual blocks. As illustrated in FIG. 6, the short residual blocks may include a plurality of convolution layers, a plurality of batch normalization layers, an activation layer (ReLU), and a summation layer.

That is, the short residual block may output a new feature map from the previous feature map (the feature map output from the (N-1)-th short attention block, where an initial feature map of a first short attention block is the previous feature map) through the convolution layer, and may sum (element-wise sum) the previous feature map and the new feature map through the summation layer. Therefore, each short residual block may sum a new feature map and the previous feature map to ensure smooth information flow between all short residual blocks of the residual block 330, thereby capable of solving the problem of feature information disappearing while extracting deep features. Meanwhile, overfitting can be prevented by applying dropout to the feature map output from the activation layer of the short residual block.

The sum block 340 may sums the focused feature map output from the attention block 320 and the deep feature map output from the residual block 330 to output a final feature map. In this case, the sum block 340 may use element-wise sum to sum the focused feature map and the final feature map.

The classification block 350 may classify the type of arrhythmia based on the final feature map output from the sum block 340. In this case, the classification block 350 may estimate the probability according to each class (arrhythmia type) based on the final feature map, and classify the class having the highest probability as the type of arrhythmia.

In an exemplary embodiment, the classification block 350 may perform global max pooling and global average pooling in parallel and simultaneously through a global max pooling layer and a global average pooling layer. In addition, the classification block 350 may perform an operation of concatenating pooling results produced, which are respectively output through the maximum pooling layer and the average pooling layer, through a concatenation layer. Through this, output data from separate layers may be combined into one. Here, by using the global max pooling layer and the global average pooling layer, the final feature map may be output as a feature map of a preset number of one-dimensional matrices. In addition, the classification block 350 may estimate the probability according to each class through a fully connected layer using the output feature map of the preset number of one-dimensional matrices, and classify the class having the highest probability as a type of arrhythmia. Here, a Softmax function, etc. may be used as the fully connected layer. Here, the preset number may be the number of types of arrhythmia.

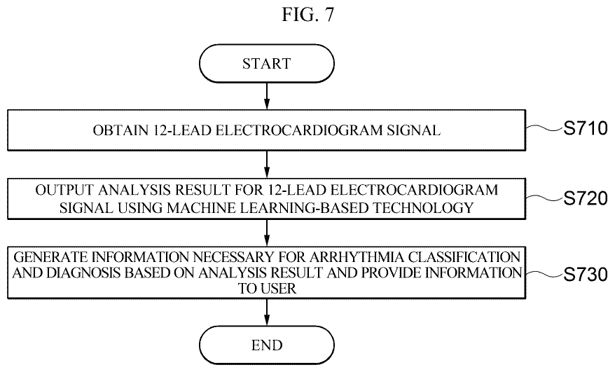

FIG. 7 is a flow chart for describing a method for providing information necessary for arrhythmia classification and diagnosis using a 12-lead electrocardiogram signal according to one embodiment of the present invention. The method illustrated in FIG. 7 may be performed by, for example, the apparatus for providing information necessary for arrhythmia classification and diagnosis using the 12-lead electrocardiogram signal described above. In the illustrated flowchart, the method is described by being divided into a plurality of steps, but at least some of the steps may be performed in a different order, combined with other steps and performed together, omitted, divided into sub-steps, or performed by being added with one or more steps (not illustrated).

The apparatus 100 for providing information necessary for arrhythmia classification and diagnosis using the 12-lead electrocardiogram signal obtains a 12-lead electrocardiogram signal (S710). For example, the apparatus 100 for providing information necessary for arrhythmia classification and diagnosis using the 12-lead electrocardiogram signal may obtain a 12-lead electrocardiogram signal measured by a medical staff through a 12-lead electrocardiogram device.

Subsequently, the apparatus 100 for providing information necessary for arrhythmia classification and diagnosis using the 12-lead electrocardiogram signal outputs an analysis result for the 12-lead electrocardiogram signal using a machine learning-based technology from the 12-lead electrocardiogram signal (S720). For example, the apparatus 100 for providing information necessary for arrhythmia classification and diagnosis using the 12-lead electrocardiogram signal may include an artificial neural network model that has been previously trained to classify arrhythmia based on the input 12-lead electrocardiogram signal.

Subsequently, the apparatus 100 for providing information necessary for arrhythmia classification and diagnosis using the 12-lead electrocardiogram signal generates information necessary for arrhythmia classification and diagnosis based on the output analysis results and provides the information to a user (e.g., a medical staff) (S730). For example, the apparatus 100 for providing information necessary for arrhythmia classification and diagnosis using the 12-lead electrocardiogram signal may display the 12-lead electrocardiogram signal through the display unit of a smart device and provide the 12-lead electrocardiogram signal to the user along with the type of arrhythmia that the patient will be diagnosed with and the probability of being diagnosed with the arrhythmia, based on the analysis results.

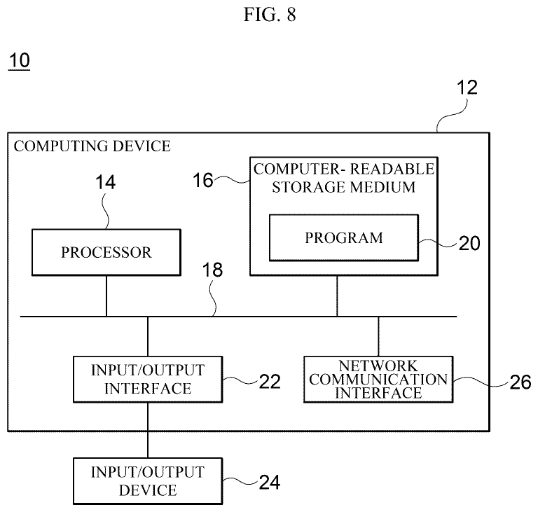

FIG. 8 is a block diagram illustrating a computing environment including a computing device according to an exemplary embodiment. In the illustrated embodiment, respective components may have different functions and capabilities other than those described below, and may include additional components in addition to those described below.

An illustrated computing environment 10 includes a computing device 12. In one embodiment, the computing device 12 may be the apparatus 100 for providing information necessary for arrhythmia classification and diagnosis using the 12-lead electrocardiogram signal.

The computing device 12 includes at least one processor 14, a computer-readable storage medium 16, and a communication bus 18. The processor 14 may cause the computing device 12 to operate according to the exemplary embodiment described above. For example, the processor 14 may execute one or more programs stored on the computer-readable storage medium 16. The one or more programs may include one or more computer-executable instructions, which, when executed by the processor 14, may be configured so that the computing device 12 performs operations according to the exemplary embodiment.

The computer-readable storage medium 16 is configured to store the computer-executable instruction or program code, program data, and/or other suitable forms of information. A program 20 stored in the computer-readable storage medium 16 includes a set of instructions executable by the processor 14. In an embodiment, the computer-readable storage medium 16 may be a memory (volatile memory such as a random access memory, non-volatile memory, or any suitable combination thereof), one or more magnetic disk storage devices, optical disk storage devices, flash memory devices, other types of storage media that are accessible by the computing device 12 and capable of storing desired information, or any suitable combination thereof.

The communication bus 18 interconnects various other components of the computing device 12, including the processor 14 and the computer-readable storage medium 16.

The computing device 12 may also include one or more input/output interfaces 22 that provide an interface for one or more input/output devices 24, and one or more network communication interfaces 26. The input/output interface 22 and the network communication interface 26 are connected to the communication bus 18. The input/output device 24 may be connected to other components of the computing device 12 through the input/output interface 22. The exemplary input/output device 24 may include a pointing device (such as a mouse or trackpad), a keyboard, a touch input device (such as a touch pad or touch screen), a speech or sound input device, input devices such as various types of sensor devices and/or photographing devices, and/or output devices such as a display device, a printer, a speaker, and/or a network card. The exemplary input/output device 24 may be included inside the computing device 12 as a component configuring the computing device 12, or may be connected to the computing device 12 as a separate device distinct from the computing device 12.

According to embodiments of the present invention, by providing information necessary for arrhythmia classification and diagnosis from a standard 12-lead electrocardiogram signal to a smart device using machine learning technology, the time required for a medical staff (cardiologist) to determine arrhythmia can be reduced.

In the above, although representative embodiments of the present disclosure have been described in detail, those skilled in the art will understand that the present disclosure may be implemented in modified forms without departing from the essential characteristics of the present disclosure. Therefore, the scope of the present disclosure is not limited to the embodiments described above, but should be defined not only by the claims described below but also by equivalents of the claims.

What is claimed is:

1. A method for providing necessary information for arrhythmia classification and diagnosis using a 12-lead electrocardiogram signal performed by a computing device including one or more processors and a memory storing one or more programs executed by the one or more processors, the method comprising:
obtaining a 12-lead electrocardiogram signal;
outputting an analysis result for the 12-lead electrocardiogram signal using a machine learning-based technology from the 12-lead electrocardiogram signal; and
generating information necessary for arrhythmia classification and diagnosis based on the analysis result.

2. The method of claim 1,
wherein, in the outputting of the classification result, the 12-lead electrocardiogram signal is received through a pre-trained artificial neural network model and a type of arrhythmia is classified based on the 12-lead electrocardiogram signal.

3. The method of claim 2,
wherein the classifying of the arrhythmia information further includes:
outputting, through an initial feature block, an initial feature map through a convolution operation based on the input 12-lead electrocardiogram signal;
outputting, through an attention block, a focused feature map through an element-wise weighting operation based on the output initial feature map;
outputting, through a residual block, a deep feature map through a shortcut operation based on the output initial feature map;
summing, through a sum block, the output focused feature map and the output deep feature map to output a final feature map; and
estimating a probability of a preset class based on the output final feature map and classifying a type of arrhythmia based on the output final feature map, through a classification block,
wherein the information required for the arrhythmia classification and diagnosis includes the type of the classified arrhythmia, the estimated probability, and the input 12-lead electrocardiogram signal.

4. The method of claim 3,
wherein the outputting of the focused feature further includes:
performing maximum pooling and average pooling in parallel on the initial feature map through a maximum pooling layer and an average pooling layer;
performing summation of pooling results, which are respectively output through the maximum pooling layer and the average pooling layer, through a first summation layer to output a weighted feature map; and
performing summation between the initial feature map and the weighted feature map through a second summation layer to output a focused feature map.

5. The method of claim 4,
wherein the residual block is configured to include N short residual blocks sequentially connected to reflect features of the initial feature map, wherein N is a natural number greater than or equal to 2, and
the N short residual blocks are configured to receive a previous feature map, which is a feature map output from an (N-1)-th short residual block, output a new feature map from the previous feature map through a convolution layer, and sum the previous feature map and the new feature map through a third summation layer to output the summation result.

6. The method of claim 5,
wherein the first summation layer and the third summation layer use element-wise sum, and
the second summation layer uses element-wise multiplication.

7. The method of claim 3,
wherein the classifying of the type of arrhythmia further includes:
performing global max pooling and global average pooling in parallel on the final feature map through a global max pooling layer and a global average pooling layer; and
performing concatenation of pooling results, which are respectively output through the global max pooling layer and the global average pooling layer, through the connection layer.

8. A computing device comprising:
one or more processors;
a memory storing one or more programs executed by the one or more processors;
a signal obtaining module configured to obtain a 12-lead electrocardiogram signal;
a signal analysis module configured to output an analysis result for the 12-lead electrocardiogram signal using a machine learning-based technology from the 12-lead electrocardiogram signal; and
a result provision module configured to generate information necessary for arrhythmia classification and diagnosis based on the analysis result.

9. The computing device of claim 8,
wherein the classification module is configured to receive the 12-lead electrocardiogram signal and is configured to be trained to classify arrhythmia information based on the 12-lead electrocardiogram signal.

10. The computing device of claim 9,
wherein the artificial neural network model includes:
an initial feature block configured to output an initial feature map through a convolution operation based on the input 12-lead electrocardiogram signal;
an attention block configured to output a focused feature map through an element-wise weighting operation based on the initial feature map output from the initial feature block;
a residual block configured to output a deep feature map through a shortcut operation based on the initial feature map output from the initial feature block;
a sum block configured to sum the focused feature map output from the attention block and the deep feature map output from the residual block to output a final feature map; and
a classification block configured to classify a type of arrhythmia based on the final feature map output from the sum block,
wherein the information required for the arrhythmia classification and diagnosis includes the type of the classified arrhythmia, the estimated probability, and the input 12-lead electrocardiogram signal.

11. The computing device of claim 10,
wherein the attention block is configured to perform maximum pooling and average pooling in parallel through a maximum pooling layer and an average pooling layer on the initial feature map output from the initial feature block, perform summation of pooling results, which are respectively output through the maximum pooling layer and the average pooling layer, through a first summation layer to output a weighted feature map, and perform summation between the initial feature map and the weighted feature map through a second summation layer to output a focused feature map.

12. The computing device of claim 11,
wherein the residual block is configured to include N short residual blocks sequentially connected to reflect features of the initial feature map output from the initial feature block, wherein N is a natural number greater than or equal to 2, and
the N short residual blocks are configured to receive a previous feature map, which is a feature map output from an (N-1)-th short residual block, output a new feature map from the previous feature map through a convolution layer, and sum the previous feature map and the new feature map through a third summation layer to output the summation result.

13. The computing device of claim 12,
wherein the first summation layer and the third summation layer use element-wise sum, and
the second summation layer uses element-wise multiplication.

14. The computing device of claim 10,
wherein the classification block is configured to perform global max pooling and global average pooling in parallel through a global max pooling layer and a global average pooling layer on the final feature map, and perform concatenation of pooling results, which are respectively output through the global max pooling layer and the global average pooling layer, through the connection layer.
